(19) United States
(12) Patent Application Publication
(10) Pub. No.: US 2010/0144873 A1
(43) Pub. Date: Jun. 10, 2010

(54) TREATMENT OF MYOCARDITIS USING FTS

(75) Inventors: Yoel Kloog, Herzliya (IL); Jakob George, Tel-Aviv (IL); Gad Keren, Kiryat Ono (IL); Rakefet Pando, Ramat-Gan (IL)

Correspondence Address: LERNER, DAVID, LITTENBERG,;KRUMHOLZ & MENTLIK, 600 SOUTH AVENUE WEST, WESTFIELD, NJ, 07090, US

(73) Assignee: RAMOT AT TEL-AVIV UNIVERSITY LTD., Tel Aviv (IL)

(21) Appl. No.: 12/595,636

(22) PCT Filed: Apr. 17, 2009
(86) PCT No.: PCT/IL08/00529

§ 371 Date: Feb. 18, 2010

Related U.S. Application Data
(60) Provisional application No. 60/923,834, filed on Apr. 17, 2007.

(51) Int. Cl. A61K 31/192; A61K 31/235; A61P 9/00

(52) U.S. Cl. 514/544; 514/568

(57) ABSTRACT

Disclosed are methods of treating myocarditis by—administering to a human in need thereof effective amounts of FTS, or various analogs thereof, or a pharmaceutically acceptable salt thereof.

This application claims the benefit of the filing date of U.S. Provisional patent Application No. 60/923,834 filed Apr. 17, 2007, the disclosure of which is hereby incorporated herein by reference.

### BACKGROUND OF THE INVENTION

Myocarditis is an inflammatory heart disease and is potentially lethal in humans. The disease is characterized by immune cell infiltration in the myocardium, with potential subsequent development of fibrosis and myocardial dysfunction [M. W. Cunningham, Am J Pathol 159:5-9 (2001)]. Myocarditis is caused by a variety of infections and systemic diseases [E. B. Lieberman, G. M. Hutchins, A. Herskowitz, et al., J Am Coll Cardiol. 18:1617-1626 (1991)]. It can become autoimmune due to cross-reactive epitopes of cardiac myosin (mostly) and infectious agents. Although the precise mechanisms for the development of myocarditis are still unresolved, its pathogenesis has been suggested to involve natural killer cells, viral-specific cytotoxic T-cells and anti-myosin antibodies [S. C. Smith, P. M. Allen, J Immunol 147:2141-7 (1991); F. L. Alvarez, N. Neu, N. R. Rose, et al., Clin Immunol Immunopathol 43:129-139 (1987); S. A. Huber, L. P. Job, J. F. Woodruff. Am J Pathol. 98:681-694 (1980)].

According to the official website of the Mayo Clinic, treatment and prognosis depend upon the underlying cause and upon the individual patient. In some cases, aggressive therapy may be necessary, such as, intravenous medications to improve the heart pumping function, placement of a pump in the aorta (intra-aortic balloon pump), use of a temporary artificial heart (assist device), and consideration of urgent heart transplantation. Some patients may have chronic and irreversible damage to the heart muscle requiring lifelong medications, while other people need medications for just a few months and then recover completely. Thus, variability in the disease makes it difficult to treat. Effective drug therapy to reverse or limit the progression of myocarditis by inhibition of the pathogenesis is not available.

### SUMMARY OF THE INVENTION

A first aspect of the present invention is directed to a method of treating myocarditis. The method comprises administering to a human in need thereof an effective amount of S-farnesylthiosalicylic acid (FTS) or an analog thereof, or a pharmaceutically acceptable salt thereof. The compound is typically administered in the form of a composition, which is formulated with at least one pharmaceutically acceptable inert ingredient (e.g., a carrier, vehicle, etc.).

While not intending to be bound to any particular theory of operation, Applicants hypothesize (as shown in the working examples herein) that FTS exerts a preventative (prophylactic) or therapeutic (treatment) effect by reducing the proliferative ability of lymphocytes, which mediate onset and progression of the disease and/or attenuate myosin-specific cellular and humoral immune responses in myocarditis patients. Accordingly, additional aspects of the present invention are directed to achieving these effects in vivo.

### DESCRIPTION OF THE DRAWINGS

FIG. 1A is a bar graph illustrating the reduced proliferation of lymphocytes in rats immunized with myosin (2.5 μg/ml) ex vivo in the presence of varying concentrations of FTS as compared to control lymphocytes. Columns, mean bars±SE. *P<0.05 compared with control.

FIG. 1B is a bar graph illustrating the dose-dependent reduction of Concavalin A-induced proliferation (4 μg/ml) of lymphocytes ex vivo in the presence of varying concentrations of FTS as compared to control lymphocytes. Columns, mean bars±SE. *P<0.05 compared with control.

FIG. 2 is a bar graph illustrating the reduced proliferation of lymphocytes in myosin-immunized Lewis rats treated with FTS as compared to control animals (PBS treated). FTS (5 mg/kg) was administered two days prior to myosin immunization and then every other day for a period of 14 days (“Protocol 1”). Columns, mean bars±SE. *P<0.05 compared with control.

FIG. 3 is a bar graph illustrating the reduced levels of IgG anti-myosin antibody (p<0.05), in vivo, according to Protocol 1 and Protocol 2 (FTS 5 mg/kg administered one week after myosin immunization for a period of 7 days) as compared to control animals (PBS-treated). Columns, mean bars±SE. P<0.05 compared with control.

FIG. 4A is a bar graph illustrating a reduction in the severity of myocarditis in Lewis rats (measured on a scale of 0-2 arbitrary units) treated according to Protocol 1 and 2 as compared with the control group (P<0.05).

FIG. 4B is a bar graph illustrating a reduction in the deposition of collagen (scar formation) in Lewis rats treated according to Protocol 1 and 2 as compared with the control group (P<0.05).

FIG. 4C are a series of macroscopic and microscopic images illustrating the results of the histological examinations across control (PBS) and experimental groups (Protocol 1 and 2). The first column illustrates a macroscopic image of the heart. The H&E stained images illustrate the microscopic examination for determining the severity of myocarditis (FIG. 4A). The Masson trichrome stained images illustrate the microscopic examination for assessing collagen deposition in the three study groups (FIG. 4B).

FIG. 5A are western immunoblot images illustrating the effect of FTS on Ras and its prominent downstream effectors in lymphocytes of FTS-treated rats in accordance with Protocol 1.

FIG. 5B is a bar graph illustrating the quantitative analysis of the immunoblots indicating reduced levels of total Ras, active Ras (Ras-GTP), p-Erk and p-Akt in lymphocytes of FTS-treated rats from the Protocol 1 group.

FIG. 6 is a table summarizing the results of the echocardiography parameters indicating improved systolic parameters such as fractional shortening and left ventricular diastolic diameter in FTS-treated rats. FTS (5 mg/kg) was administered two days prior to myosin immunization and then every other day for a period of six weeks.

### DETAILED DESCRIPTION

FTS and its analogs useful in the present invention are represented by formula I:

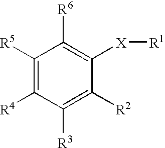

wherein
R¹ represents farnesyl or geranyl-geranyl;
R² is COOR⁷, or CONR⁷R⁸, wherein R⁷ and R⁸ are each independently hydrogen, alkyl or alkenyl;
R³, R⁴, R⁵ and R⁶ are each independently hydrogen, alkyl, alkenyl, alkoxy, halo, trifluoromethyl, trifluoromethoxy, or alkylmercapto; and
X represents S.

The structure of FTS is as follows:

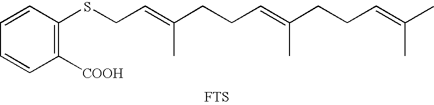

FTS analogs embraced by formula I, and which may be suitable for use in the present invention, include 5-fluoro-FTS, 5-chloro-FTS, 4-chloro-FTS, S-farnesyl-thiosalicylic acid methyl ester (FTSME), and S-geranyl, geranyl-thiosalicylic acid (GGTS). Structures of these compounds are set forth below.

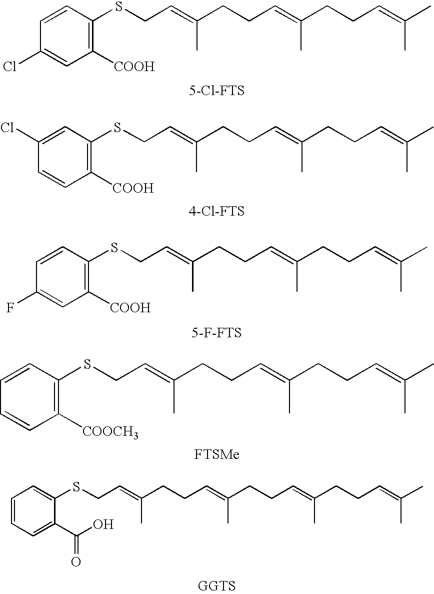

In some embodiments, GGTS is administered in an amount effective to treat a patient diagnosed with myocarditis.

Methods for preparing the compounds of formula I are disclosed in U.S. Pat. Nos. 5,705,528 and 6,462,086. See also, Marom, M., Haklai, R., Ben-Baruch, G., Marciano, D., Egozi, Y., Kloog, Y., J Biol Chem 270:22263-70 (1995).

Pharmaceutically acceptable salts of the Ras antagonists of formula I may be useful. These salts include, for example, sodium and potassium salts. Other pharmaceutically acceptable salts may be selected in accordance with standard techniques as described in Berge, S. M., Bighley, L. D., and Monkhouse, D. C., J. of Pharm. Sci. 66(1):1-19 (1977). In preferred embodiments, however, FTS and its analogs are not administered in the form of a salt (i.e., they are administered in non-salified form).

As used herein, the term “effective amount” refers to the dosage(s) of FTS that is effective for the treating, and thus includes dosage amounts that ameliorate symptom(s) of myocarditis and its associated manifestations, diminish extent of disease, delay or slow disease progression, or achieve partial or complete remission or prolong survival. The average daily dose of FTS generally ranges from about 50 mg to about 2000 mg, and in some embodiments, ranges from about 200 mg to about 1600 mg. According to Phase I human clinical trials (for various cancers) conducted by Concordia Pharmaceuticals, Inc., S-Farnesylthiosalicylic acid (FTS, Salirasib) is a relatively safe compound with no dose-limiting toxicities at doses up to 1800 mg/day.

The frequency of administration, dosage amounts, and the duration of treatment using FTS may be determined depending on several factors, e.g., the overall health, size and weight of the patient, the severity of the disease, the patient's tolerance to the treatment, and the particular treatment regimen being administered. For example, duration of treatment with FTS may last a day, a week, a year, or until remission of the disease is achieved. In some embodiments, FTS is administered on a daily basis, e.g., each in single once-a-day or divided doses. Thus, relative timing of administration of FTS is not critical.

The methods of the present invention may be used for the treatment of myocarditis in humans. In preferred embodiments, FTS is administered orally. In an oral dosage form, the FTS is typically present in a range of about 100 mg to about 500 mg, and in some embodiments, from about 100 mg to about 300 mg.

In some embodiments, FTS may be administered by dosing orally on a daily basis for three or four weeks, followed by a one-week “off period”, and repeating until remission is achieved. In another embodiment, FTS may be administered by dosing twice daily and continuing the treatment until remission is achieved. Parenteral administration may also be suitable.

In another embodiment, the treatment regimen may entail administration with oral FTS (e.g., a capsule or a tablet) continuously without interruption (i.e., without an “off period”). Thus, dosing regimens for administering FTS may be adjusted to meet the particular needs of the patient.

Oral compositions for FTS and its analogs for use in the present invention can be prepared by bringing the agent(s) into association with (e.g., mixing with) a pharmaceutically acceptable carrier or vehicle (e.g., a pharmaceutically acceptable inert ingredient). Suitable carriers are selected based in part on the mode of administration. Carriers are generally solid or liquid. In some cases, compositions may contain solid and liquid carriers. Compositions suitable for oral administration that contain the active are preferably in solid dosage forms such as tablets (e.g., including film-coated, sugar-coated, controlled or sustained release), capsules, e.g., hard gelatin capsules (including controlled or sustained release) and soft gelatin capsules, powders and granules. The compositions, however, may be contained in other carriers that enable administration to a patient in other oral forms, e.g., a liquid or gel. Regardless of the form, the composition is divided into individual or combined doses containing predetermined quantities of the active ingredient or ingredients.

Oral dosage forms may be prepared by mixing the active pharmaceutical ingredient or ingredients with one or more appropriate carriers (optionally with one or more other pharmaceutically acceptable additives or excipients), and then formulating the composition into the desired dosage form e.g., compressing the composition into a tablet or filling the composition into a capsule or a pouch. Typical carriers and excipients include bulking agents or diluents, binders, buffers or pH adjusting agents, disintegrants (including crosslinked and super disintegrants such as croscarmellose), glidants, and/or lubricants, including lactose, starch, mannitol, microcrystalline cellulose, ethylcellulose, sodium carboxymethylcellulose, hydroxypropylmethylcellulose, dibasic calcium phosphate, acacia, gelatin, stearic acid, magnesium stearate, corn oil, vegetable oils, and polyethylene glycols. Coating agents such as sugar, shellac, and synthetic polymers may be employed, as well as colorants and preservatives. See, Remington's Pharmaceutical Sciences, The Science and Practice of Pharmacy, 20th Edition, (2000).

Liquid form compositions include, for example, solutions, suspensions, emulsions, syrups, and elixirs. The active ingredient or ingredients, for example, can be dissolved or suspended in a pharmaceutically acceptable liquid carrier such as water, an organic solvent (and mixtures thereof), and/or pharmaceutically acceptable oils or fats. Examples of liquid carriers for oral administration include water (particularly containing additives as above, e.g., cellulose derivatives, preferably in suspension in sodium carboxymethyl cellulose solution), alcohols (including monohydric alcohols (including monohydric alcohols and polyhydric alcohols, e.g., glycerin and non-toxic glycols) and their derivatives, and oils (e.g., fractionated coconut oil and arachis oil). The liquid composition can contain other suitable pharmaceutical additives such as solubilizers, emulsifiers, buffers, preservatives, sweeteners, flavoring agents, suspending agents, thickening agents, colorants, viscosity regulators, stabilizers or osmoregulators.

Carriers suitable for preparation of compositions for parenteral administration include Sterile Water for Injection, Bacteriostatic Water for Injection, Sodium Chloride Injection (0.45%, 0.9%), Dextrose Injection (2.5%, 5%, 10%), Lactated Ringer's Injection, and the like. Dispersions can also be prepared in glycerol, liquid polyethylene glycols and mixtures thereof, and in oils. Compositions may also contain tonicity agents (e.g., sodium chloride and mannitol), antioxidants (e.g., sodium bisulfite, sodium metabisulfite and ascorbic acid) and preservatives (e.g., benzyl alcohol, methyl paraben, propyl paraben and combinations of methyl and propyl parabens).

In order to fully illustrate the present invention and advantages thereof, the following specific examples/experiments are given, it being understood that the same is intended only as illustrative and in no way limitative.

### Example 1

### Experimental Design

The purpose of these in vivo, ex vivo, and in vitro experiments was to assess the ability of FTS to suppress the progression of myocarditis and subsequent myocardial dysfunction. Here, the effects of the Ras inhibitor FTS on a rodent model of experimental autoimmune myocarditis (EAM) were examined. In addition, ex vivo studies of FTS on lymphocytes excised from the myosin-induced EAM rodent models were performed. Furthermore, the influence of FTS on the humoral response to myosin was evaluated. In other experiments, excised heart sections were examined using microscopy and staining techniques. In another experiment, lymphocytes excised from the rodent models from control and FTS-treated rats were examined using quantitative analysis of western immunoblots. Additionally, functional improvements using cardiography were investigated in EAM rodent models. The primary goal was to determine: (I) the effect of FTS on proliferation of myosin-treated lymphocytes ex vivo; (II) the effect of FTS on proliferation of myosin-treated lymphocytes in vivo; (III) the effect of FTS on IgG anti-myosin antibody levels; (IV) the effect of FTS on scar area formation and the severity of myocarditis in animal models; (V) the effect of FTS on Ras-signaling pathways in lymphocytes of FTS-treated rodent models; and (VI) the effect of FTS in preserving myocardial function.

Overall, the results of the experiments described herein indicated that FTS, given as treatment or prevention therapy, was capable of ameliorating the progression of EAM. The beneficial effect of FTS was associated with a reduction in the cellular and humoral immune responses to myosin. Functional improvements were also observed.

The results of the first set of experiments (I) demonstrated that FTS (25 and 50 μM) significantly reduced lymphocyte proliferation in rats after myosin immunization as compared to controls (p<0.05). FTS (25 and 50 μM) also dose-dependently reduced Concavalin A-induced proliferation. Thus, FTS treatment ex vivo showed a reduction in myosin-induced lymphocyte proliferation, and a dose-dependent reduction in proliferation in positive controls [Concavalin A (4 μg/ml)].

The second set of experiments (II) revealed that FTS administration according to Protocol 1 (prevention regimen) reduced the proliferative ability of myosin-induced lymphocytes as compared to control animals. Thus, FTS attenuated the proliferation of myosin-induced lymphocytes in vivo.

In the third set of experiments (III), FTS injections according to Protocol 1 and 2 (prevention and treatment regimens) reduced IgG anti-myosin antibody levels in comparison with controls. Thus, FTS was associated with a reduction in humoral responses to myosin in vivo.

In the fourth set of experiments (IV), microscopic evaluations of H&E stained heart tissue revealed that FTS administration according to Protocol 1 (prevention regimen) reduced the severity of myocarditis as compared with controls. Results also showed that FTS injections according to Protocol 2 (treatment regimen) reduced the severity of myocarditis as compared with controls. Additionally, microscopic assessments of collagen deposition (scar formation) revealed that FTS administration attenuated myocardial damage induced by EAM under both protocols as compared with controls.

In the fifth set of experiments (V), densitometric analysis of the immunoblots revealed that FTS administration according to Protocol 1 (prevention regimen) reduced the levels of total Ras, active Ras (Ras-GTP), p-Erk and p-Akt in lymphocytes. In addition, levels of Ras-GTP were reduced in lymphocytes of FTS-treated rats compared with control group (p<0.05). Levels of total Ras also decreased in lymphocytes of FTS-treated rats compared with control group. Levels of p-Erk decreased in lymphocytes of FTS treated rats compared with the control group (p<0.05), although levels of total Erk did not differ in both groups. Furthermore, FTS treatment was associated with a decrease in the levels of p-Akt as compared with the control group (p<0.05), while levels of total Akt did not differ in both groups.

In the sixth set of experiments (VI), thoracic echocardiographs revealed that prolonged treatment with FTS improved systolic parameters, such as fractional shortening and left ventricular diastolic diameter. Thus, treatments with FTS administered under a prevention regimen and continued for six weeks significantly attenuated myocardial damage induced by EAM.

### Materials and Methods

### Animals

Male Lewis rats were purchased from Harlan and maintained in a local vivarium under conventional conditions.

### Induction of EAM

Porcine cardiac myosin (Sigma) was dissolved in PBS to a concentration of 0.4 μg/ul. Each rat was immunized by a single subcutaneous injection in both footpads on day 0 with an emulsion containing 1 mg of cardiac myosin with an equal volume of complete Freund's adjuvant containing 10.0 mg/mL of heat-killed Mycobacterium tuberculosis.

### FTS Preparation for Intraperitoneal Administration

FTS was provided by Concordia Pharmaceuticals, Inc. (Ft. Lauderdale, Fla.). FTS was stored in chloroform, which was evaporated under a stream of nitrogen immediately before use. The powder was dissolved in absolute ethanol and diluted to the desired concentration in sterile PBS made basic with NaOH. Carrier solution (1000 μl) containing 1.35 mg of FTS (5 mg/kg) was injected intraperitoneally (i.p.) into each rat. Control solution was prepared at the same time, starting with PBS and absolute ethanol.

### FTS Intraperitoneal Administration

Two groups of rats were induced to develop EAM by immunization with cardiac myosin according to two protocols. In Protocol 1 (prevention regimen), FTS or PBS (control) was administered two (2) days prior to myosin immunization at a dose of 5 mg/kg (FTS pre-treatment before immunization). Thereafter, dosing was continued for a period of fourteen (14) days with administration of FTS or PBS occurring every other day. In Protocol 2 (treatment regimen), FTS or PBS (control) was administered fourteen (14) days after porcine cardiac myosin immunization at a dose of 5 mg/kg (FTS post-EAM). Thereafter, dosing was continued for a period of seven (7) days with administration of FTS or PBS occurring every other day. Under both protocols, the Lewis rats were sacrificed at day 21 and hearts, lymph nodes and spleens were excised for histological and immunological analyses.

Another group of experiments was designed to study the effects of FTS on prevention of myocardial dysfunction following myosin-induced EAM. Under this regimen, FTS or PBS was given two (2) days before myosin immunization, then every other day for six (6) weeks (when compromised heart function is expected). After six (6) weeks, M-mode echocardiography was performed as described in George J, Biner S, Keren P, Barshack I, Goldberg I, Sherez J, Levitski A, Keren G, Roth A. Exp Mol. Pathol. 74(3):314-8 2003.

### Lymphocytes Proliferation Assay

Lymphocytes were obtained from lymph nodes of FTS- and PBS-treated Lewis rats. The cells were washed and 1×10⁵ cells/well seeded in a 96 microwell plate in RPMI medium. Cardiac myosin at 2.5 μg/ml and 5 μg/ml was added to the medium. Concavalin A (4 μg/ml was used as a positive control for lymphocyte proliferation.

Lymphocyte proliferation was evaluated using XTT reagent, according to standard procedures, after 96 h of incubation.

### Determination of Ras, Erk, P-Erk, Akt and P-Akt

Lymphocytes were obtained from lymph nodes of FTS- and PBS-treated rats. The lymphocytes were homogenized in cold homogenization buffer containing protease inhibitors. Protein concentration was determined by the Bradford assay and samples containing 100 μg protein were used for determination of total Ras, total Erk, P-Erk, total Akt and P-Akt by Western immunoblotting using, respectively: pan-anti-Ras Ab (Ab03; Santa Cruz, Calif.), anti Erk Ab (Santa Cruz, Calif.), anti P-Erk Ab (Sigma), anti Akt Ab (Cell Signaling), and anti P-Akt Ab (Cell Signaling).

Enhanced chemiluminescence (ECL) and densitometric analysis were performed as detailed in R. Haklai, M. G. Weisz, G. Elad, et al., Biochemistry 37:1306-1314 (1998).

### Determination of Ras-GTP

The lymphocytes were prepared as mentioned above. Samples containing 1.5 mg protein used for determination of levels of active GTP-bound Ras by the glutathione S-transferase-RBD pull-down assay followed by Western immunoblotting with pan-anti-Ras Ab as detailed in G. Elad-Sfadia, R. Haklai, E. Ballan et al., J. Biol. Chem. 277:37169-75 (2002).

### Histopathology

Hearts were removed and fixed in 10% formalin, then embedded in paraffin. Several transverse sections were cut from the paraffin-embedded samples and stained with Hematoxylin and Eosin (H&E). Every fifth section was examined for the presence of myocarditis by light microscopy, and was evaluated according to previously published criteria (the Dallas criteria) describing the severity of myocarditis as follows: severe (2)>50% of the heart involved; moderate (1)=10-50% involved; and minimal (0)=<10% involved, or normal.

Sections from each heart were also stained with Masson trichrome for an assessment of scar area (collagen deposition).

### FTS Preparation for Ex Vivo Assay

FTS was provided by Concordia Pharmaceuticals, Inc. (Ft. Lauderdale, Fla.). FTS was stored in chloroform, which was evaporated under a stream of nitrogen immediately before use. The powder was dissolved in DMSO. This solution was diluted with RPMI/10% FCS to yield a 10 mM drug stock solution containing 10% DMSO. A portion of this solution was applied to the cells at a dilution of 1:100.

Ex Vivo Proliferation Assay with FTS

Five (5) rats with EAM were sacrificed on day fourteen (14). Lymphocytes were taken from lymph nodes, prepared as described and 1×10⁵ cells/well seeded in 96 microwell plates.

Lymphocytes were incubated in the presence of Myosin 2.5 μg/ml or Concavalin A in the presence of 0, 3.125, 6.25, 12.5, and 25 μM FTS. Lymphocyte proliferation was evaluated using XTT reagent after 96 h of incubation.

### IgG Anti-Myosin Antibodies

Sera obtained from all sacrificed rats was studied for anti-myosin IgG antibody levels by ELISA as described in J. George, A. Adler, I. Barshack, et al., Cardiovasc Pathol. 13:221-419 (2004)].

### Statistical Analysis

Results are expressed as mean±SE. Differences between groups were compared using student's t-test. Differences were considered significant at P≦0.05.

### Results

### I. FTS Attenuated the Proliferation of Myosin-Treated and Concavalin A-Treated Lymphocytes Ex Vivo.

Experimental autoimmune myocarditis (EAM) is a potentially useful animal model of myocarditis, induced by immunization of rats or mice with cardiac myosin. EAM in the rat follows a severe course that resembles the fulminant form of human myocarditis [M. Kodama, Y. Matsumoto, M. Fujiwara, et al., Clin Immunol Immunopathol. 57:250-262 (1990)]. The onset of EAM occurs about ten (10) days after immunization and the disorder is transferred by T-cells. Here, we examined the impact of FTS on proliferation of myosin-treated lymphocytes ex vivo.

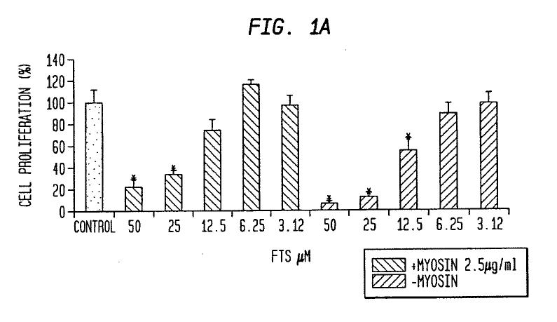

To investigate the effect of FTS on the proliferative capacity of lymphocytes from EAM rats against myosin ex vivo, lymph nodes were excised from the EAM rodent models fourteen (14) days following myosin immunizations. Thereafter, lymphocytes were incubated in the presence of (A) myosin (2.5 μg/ml) and (B) Concavalin A (4 μg/ml) with varying concentrations of FTS. FTS (25 and 50 μM) significantly reduced lymphocyte proliferation to myosin (79±7%) as compared to controls (p<0.05) (FIG. 1A). In addition, lymphocytes treated with Concavalin A also showed a dose-dependent reduction in proliferation as compared with controls. The proliferation rate was reduced in the presence of 50 μM FTS ex vivo by 48% (FIG. 1B). Thus, proliferation of lymphocytes against potential cardiac epitopes may be attenuated following FTS administration.

### II. FTS Attenuated the Proliferation of Myosin-Treated Lymphocytes In Vivo.

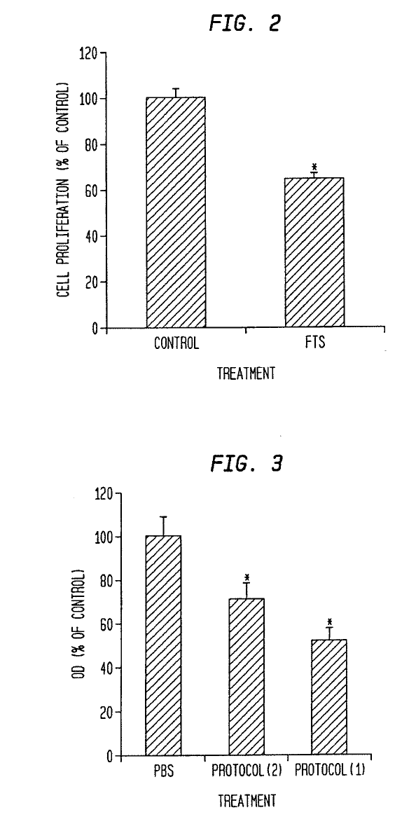

Next, to determine the effects of in vivo treatment with FTS on lymphocyte proliferation, two protocols were designed. In Protocol 1 (prevention regimen), FTS or PBS (control) was administered two (2) days prior to myosin immunization at a dose of 5 mg/kg (FTS pre-treatment before immunization). Thereafter, dosing was continued for a period of fourteen (14) days with administration of FTS or PBS occurring every other day. In Protocol 2 (treatment regimen), FTS or PBS (control) was administered fourteen (14) days after porcine cardiac myosin immunization at a dose of 5 mg/kg (FTS post-EAM). Thereafter, dosing was continued for a period of seven (7) days with administration of FTS or PBS occurring every other day. Under both protocols, the Lewis rats were sacrificed at day twenty-one (21) and the organs were excised for further analysis. FTS administered in accordance with Protocol 1 resulted in a significant decrease (˜35%) in lymphocyte proliferation in comparison with PBS (p<0.05) (FIG. 2). Moreover, the proliferation in response to the non-specific mitogen Concavalin A was not diminished in FTS-treated rats, as compared to control animals (data not show).

### III. FTS Reduced IgG Anti-Myosin Antibody Levels In Vivo.

To evaluate the influence of FTS on the humoral response to myosin, IgG anti-myosin levels were examined under two protocols. FTS administered in accordance with Protocol 1 led to significantly reduced levels of IgG anti-myosin antibody (p<0.05). In addition, FTS administered in accordance with Protocol 2 also significantly reduced IgG anti-myosin antibody levels in the FTS-treated rats (p<0.05) (FIG. 3). Thus, in both prevention and in treatment protocols, FTS induced a reduction in IgG anti-myosin antibody levels in comparison with the control group. These results are consistent with the findings in the foregoing experiments demonstrating that lymphocyte proliferation to myosin was diminished by FTS treatment in vivo and ex vivo.

### IV. FTS Attenuated the Severity of Myocarditis and Subsequent Myocardial Dysfunction.

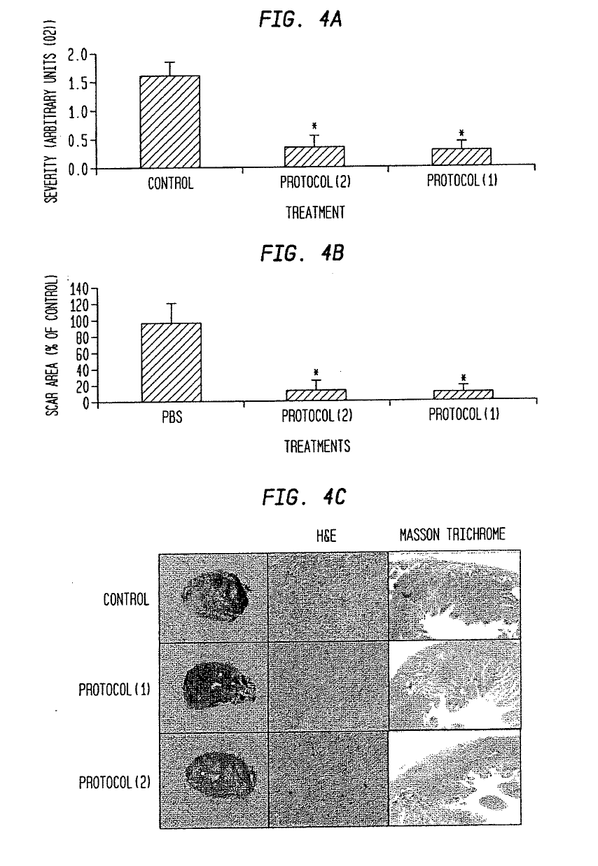

To determine the effect of FTS on the severity of myocarditis and subsequent myocardial dysfunction, histological examinations of the hearts from all experimental groups were conducted using macroscopy and microscopy. First, to determine the severity of myocarditis, heart sections were stained with H&E and the myocarditis score was evaluated using a scale of 0-2 arbitrary units (the Dallas criteria). Rats that received FTS according to Protocol 1 (prevention regimen) showed a significant reduction in the severity of myocarditis 0.28±0.15 compared with control group 1.61±0.23 in the control (p<0.05) (FIG. 4A). Rats that received FTS according to Protocol 2 also showed a significant reduction in the severity of myocarditis 0.33±0.24 compared with control group (p<0.05) (FIG. 4A).

In order to evaluate the severity of scaring of the heart tissue, Masson trichrome staining was performed to assess collagen deposition in the three study groups. Extensive deposition of collagen was observed in the control rats. FTS-treated rats from both protocols (Protocol 1 & 2) exhibited reduced collagen deposition. FIGS. 4B and C illustrate typical representations of the stained sections and quantitative analysis of the results.

### V. FTS Caused a Significant Decrease In the Downstream Effectors Phospho-Akt and Phospho-Erk, While Total Erk and Total Akt Were Not Affected.

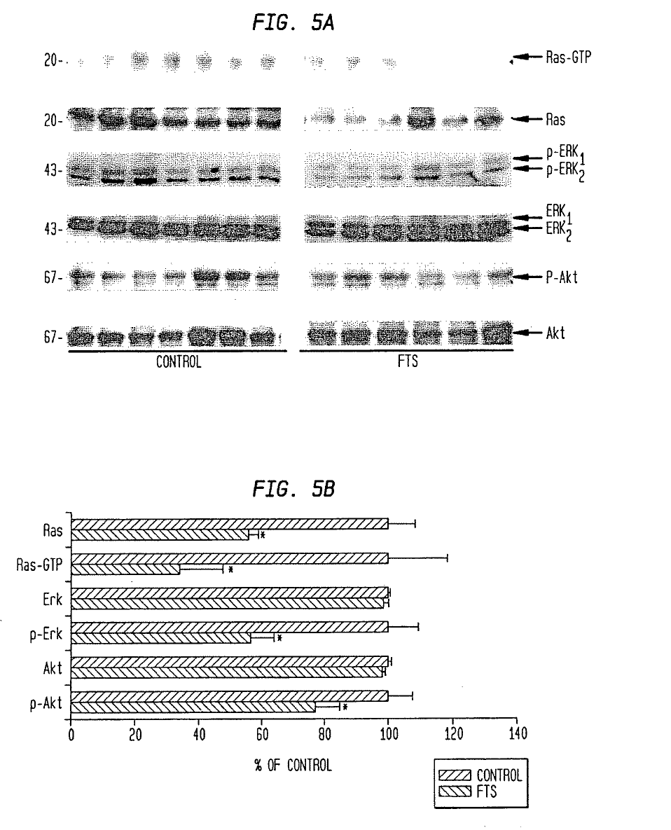

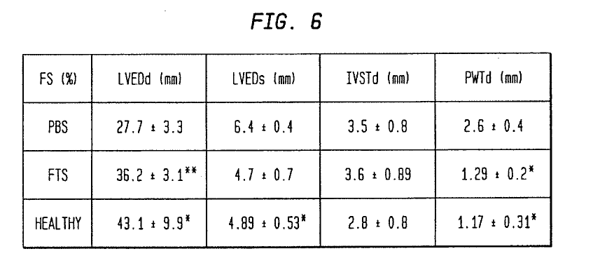

To determine the effects of FTS on Ras and its downstream signaling effectors in lymphocytes from treated rats, western immunoblotting analysis was performed. The results presented in FIGS. 5A and 5B show that FTS downregulated total Ras and Ras-GTP in lymphocytes from treated rats as compared to control animals. These results indicate that FTS did not significantly alter the ratio of Ras-GTP to total Ras. FTS not only decreased Ras protein levels, but also had a functional effect on Ras signaling activity. FTS caused a significant decrease in the downstream effectors phospho-Akt and phospho-Erk, while total Erk and total Akt were not affected by FTS. This reduction is relevant since Erk and Akt signaling pathway are known to promote cell cycle progression and survival, enabling lymphocytes to thrive and become antigen-responsive.

### VI. Prolonged Treatment With FTS Significantly Attenuated Myocardial Damage Induced by Experimental Autoimmune Myocarditis In Vivo.

To determine the effects of prolonged FTS treatment on the prevention of myocardial dysfunction induced following EAM, transthoracic echocardiography was performed. Under a preventative treatment regimen, FTS or PBS (control) was administered two (2) days prior to myosin immunization, and then every other day in the course of six (6) weeks (when compromised heart function is expected). After six (6) weeks, M-mode echocardiography was performed. Results revealed improved systolic parameters such as fractional shortening and left ventricular diastolic diameter in FTS-treated rats as compared with PBS-treated and healthy animals (FIG. 6). Thus, prolonged treatment with FTS significantly attenuated myocardial damage associated with EAM in vivo.

The publications cited in the specification, patent publications and non-patent publications, are indicative of the level of skill of those skilled in the art to which this invention pertains. All of these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of treating a human afflicted with myocarditis comprising administering to the human an effective amount of farnesylthiosalicylic acid (FTS) or an analog thereof as represented by the formula: 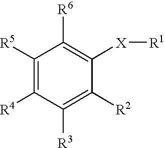
wherein
R¹ represents farnesyl or geranyl-geranyl;
R² is COOR⁷, or CONR⁷R⁸, wherein R⁷ and R⁸ are each independently hydrogen, alkyl or alkenyl;
R³, R⁴, R⁵ and R⁶ are each independently hydrogen, alkyl, alkenyl, alkoxy, halo, trifluoromethyl, trifluoromethoxy, or alkylmercapto; and
X represents S; or a pharmaceutically acceptable salt thereof.

2. The method of claim 1, wherein the human is administered FTS.

3. The method of claim 1, wherein the human is administered an analog of FTS which is GGTS.

4. The method of claim 1, wherein FTS or its analog or a pharmaceutically acceptable salt thereof is administered orally.

5. The method of claim 1, wherein FTS or its analog or a pharmaceutically acceptable salt thereof is administered intraperitoneally.

6. The method of claim 1, wherein the FTS or its analog or a pharmaceutically acceptable salt is formulated with at least one carrier.

7. The method of claim 1, wherein the human is administered an effective amount from about 50 mg to about 2000 mg per day.

8. The method of claim 7, wherein the human is administered an effective amount from about 200 mg to about 1600 mg per day.

9. The method of claim 1, wherein the treatment is prophylactic.

10. A method for reducing the proliferative ability of lymphocytes comprising administering to the human an effective amount of farnesylthiosalicylic acid (FTS) or an analog thereof as represented by the formula: 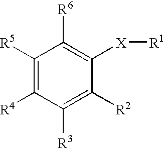
wherein
R¹ represents farnesyl or geranyl-geranyl;
R² is COOR⁷, or CONR⁷R⁸, wherein R⁷ and R⁸ are each independently hydrogen, alkyl or alkenyl;
R³, R⁴, R⁵ and R⁶ are each independently hydrogen, alkyl, alkenyl, alkoxy, halo, trifluoromethyl, trifluoromethoxy, or alkylmercapto; and
X represents S; or a pharmaceutically acceptable salt thereof.

11. The method of claim 10, wherein the human is administered FTS.

12. The method of claim 10, wherein the human is administered an analog of FTS which is GGTS.

13. The method of claim 10, wherein FTS or its analog or a pharmaceutically acceptable salt thereof is administered orally.

14. The method of claim 10, wherein FTS or its analog or a pharmaceutically acceptable salt thereof is administered intraperitoneally.

15. The method of claim 10, wherein the FTS or its analog or a pharmaceutically acceptable salt is formulated with at least one carrier.

16. The method of claim 10, wherein the human is administered an effective amount from about 50 mg to about 2000 mg per day.

17. The method of claim 16, wherein the human is administered an effective amount from about 200 mg to about 1600 mg per day.

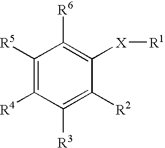

18. A method of treating a human afflicted with myocarditis comprising orally administering to the human an effective amount of S-farnesylthiosalicylic acid.

19. The method of claim 18, wherein the human is administered an effective amount from about 50 mg to about 2000 mg per day.

20. The method of claim 19, wherein the human is administered an effective amount from about 200 mg to about 1600 mg per day.
